(19) United States
(12) Patent Application Publication
(10) Pub. No.: US 2020/0214824 A1
(43) Pub. Date: Jul. 9, 2020

(54) VASCULAR GRAFT PROTECTOR

(71) Applicant: RELIANTHEART, INC., HOUSTON, TX (US)

(72) Inventors: BRYAN E LYNCH, HOUSTON, TX (US); FREDERICK D SWAIN, HOUSTON, TX (US)

(21) Appl. No.: 16/628,952

(22) PCT Filed: Jun. 30, 2018
(86) PCT No.: PCT/US18/40522

§ 371 Date: Jan. 6, 2020

Related U.S. Application Data
(60) Provisional application No. 62/527,790, filed on Jun. 30, 2017.

(51) Int. Cl. A61F 2/07; A61B 17/11

(52) U.S. Cl. CPC A61F 2/07; A61M 1/1008; A61B 17/11

(57) ABSTRACT

A vascular graft protector configured for removal from ingrown tissue comprising an axial spine from which emanate a plurality of semi-circular circumferential rib sets having ends separated by a circumferential gap and wherein each rib set is separated by an axial gap.

### CROSS REFERENCE TO RELATED APPLICATIONS

This application claims priority to and benefit of U.S. Provisional Application No. 62/527,790, filed on Jun. 30, 2017, the entire contents and disclosure of which is incorporated herein by reference for all purposes.

### BACKGROUND OF THE INVENTION

### Field of the Invention

The inventions disclosed and taught herein relate generally to vascular graft protectors; and more specifically related to vascular graft protectors that facilitate removal from ingrown tissue.

### Description of the Related Art

The inventions disclosed and taught herein are directed to vascular graft protectors that facilitate removal from ingrown tissue.

### BRIEF SUMMARY OF THE INVENTION

The following are brief summaries of various non-limiting embodiments of the inventions disclosed herein.

A vascular graft protector comprising, a hollow cylindrical body defined by a plurality of sets of semi-circular circumferential primary ribs emanating from an axial spine; and wherein each primary rib set has first and second ends separated by a circumferential gap.

A vascular graft protector comprising, a hollow cylindrical body defined by a plurality of sets of semi-circular circumferential primary ribs emanating from an axial spine; wherein each primary rib set has first and second ends separated by a circumferential gap; and an axial gap separating adjacent primary ribs.

A vascular graft protector comprising a hollow cylindrical body defined by a plurality of sets of semi-circular circumferential primary ribs emanating from an axial spine; wherein each primary rib set has first and second ends separated by a circumferential gap; and an axial gap separating adjacent primary ribs wherein the axial gap separating adjacent primary rib sets has an axial length of between about 0.5 times and about 3 times a width of the primary rib.

A vascular graft protector comprising a hollow cylindrical body defined by a plurality of sets of semi-circular circumferential primary ribs emanating from an axial spine, wherein each primary rib set has first and second ends separated by a circumferential gap; an axial gap separating adjacent primary ribs, and a termination rib set at each end of the graft protector wherein each termination rib set has a width greater than a width of a primary rib.

A vascular graft protector comprising a hollow cylindrical body defined by a plurality of sets of semi-circular circumferential primary ribs emanating from an axial spine; wherein each primary rib set has first and second ends separated by a circumferential gap, and a guide channel along the length of the axial spine configured to guide a scalpel.

A vascular graft protector comprising, a hollow cylindrical body defined by a plurality of sets of semi-circular circumferential primary ribs emanating from an axial spine; wherein each primary rib set has first and second ends separated by a circumferential gap; and at least one termination rib set located between ends of the graft protector, wherein the at least one termination rib set has a width greater than a width of a primary rib.

A vascular graft protector comprising a hollow cylindrical body defined by a plurality of sets of semi-circular circumferential primary ribs emanating from an axial spine, wherein each primary rib set has first and second ends separated by a circumferential gap; an axial gap separating adjacent primary ribs, a termination rib set at each end of the graft protector and at least one termination rib set located between ends of the graft protector, wherein each termination rib set has a width greater than a width of a primary rib.

A vascular graft protector comprising, a hollow cylindrical body defined by a plurality of sets of semi-circular circumferential primary ribs emanating from an axial spine; wherein each primary rib set has first and second ends separated by a circumferential gap; and an interlock system between one or more primary rib sets configured to prevent first and second ends from sliding over or past one another.

A vascular graft protector comprising, a hollow cylindrical body defined by a plurality of sets of semi-circular circumferential primary ribs emanating from an axial spine; wherein each primary rib set has first and second ends separated by a circumferential gap; and an interlock system between each primary rib set configured to prevent each first and second ends from sliding over or past one another.

A vascular graft protector comprising, a hollow cylindrical body defined by a plurality of sets of semi-circular circumferential primary ribs emanating from an axial spine; wherein each primary rib set has first and second ends separated by a circumferential gap; and at least one termination rib set located between ends of the graft protector, wherein the at least one termination rib set has a width greater than a width of a primary rib; and an interlock system between each primary rib set and termination rib set configured to prevent first and second ends from sliding over or past one another.

A vascular graft protector comprising a hollow cylindrical body defined by a plurality of sets of semi-circular circumferential primary ribs emanating from an axial spine, wherein each primary rib set has first and second ends separated by a circumferential gap; an axial gap separating adjacent primary ribs, a termination rib set at each end of the graft protector and at least one termination rib set located between ends of the graft protector, wherein each termination rib set has a width greater than a width of a primary rib; and an interlock system between each primary rib set and termination rib set configured to prevent first and second ends from sliding over or past one another.

A vascular graft protector comprising, a hollow cylindrical body defined by a plurality of sets of semi-circular circumferential primary ribs emanating from an axial spine; wherein each primary rib set has first and second ends separated by a circumferential gap; wherein each primary rib set is separated from an adjacent primary rib set by an axial gap; one or more termination rib sets located along the length of the protector wherein each termination rib has a width greater than a width of a primary rib; a guide channel along the length of the axial spine configured to guide a scalpel to dissect ingrown tissue; and an interlock system associated with one or more ends of primary and termination rib sets and configured to minimize the ends sliding over or past one another.

A vascular graft protector comprising, a hollow cylindrical body defined by a plurality of sets of semi-circular circumferential primary ribs emanating from an axial spine; wherein each primary rib set has first and second ends separated by a circumferential gap; wherein each primary rib set is separated from an adjacent primary rib set by an axial gap; one or more termination rib sets located along the length of the protector wherein each termination rib has a width greater than a width of a primary rib; a guide channel along the length of the axial spine configured to guide a scalpel to dissect ingrown tissue; and an interlock system associated with one or more ends of primary and termination rib sets and configured to minimize the ends sliding over or past one another; and the axial gap is between about 0.5 times and about 3 times a width of the primary rib.

A method of removing the vascular graft protector comprising dissecting ingrown tissue surrounding the vascular graft protector along the axial spine; and pulling the vascular graft protector free of the ingrown tissue.

A method of removing the vascular graft protector of claim 12 comprising dissecting ingrown tissue surrounding the vascular graft protector along the axial spine by sliding a scalpel along the guide channel; and pulling the vascular graft protector free of the ingrown tissue.

### BRIEF DESCRIPTION OF THE SEVERAL VIEWS OF THE DRAWINGS

The following figures form part of the present specification and are included to further demonstrate certain aspects of the present invention. The invention may be better understood by reference to one or more of these figures in combination with the detailed description of specific embodiments presented herein.

FIGS. 1 and 2 illustrate a conventional vascular graft protector in use with a left ventricular assist device.

FIG. 3 illustrates a vascular graft protector according to the disclosed inventions.

FIG. 4 illustrates another view of the vascular graft protector illustrated in FIG. 3.

FIG. 5 illustrates a close-up view of another embodiment of a vascular graft protector according to the disclosed inventions.

While the inventions disclosed herein are susceptible to various modifications and alternative forms, only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### DETAILED DESCRIPTION

The Figures described above and the written description of specific structures and functions below are not presented to limit the scope of what Applicants have invented or the scope of the appended claims. Rather, the Figures and written description are provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill in this art having benefit of this disclosure. It must be understood that the inventions disclosed and taught herein are susceptible to numerous and various modifications and alternative forms. Lastly, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the appended claims.

Applicant has invented a flexible vascular graft protector comprising a plurality of semicircular circumferential ribs emanating from an axial spine configured to facilitate removal from ingrown tissue. By semicircular, Applicant means that the rib does not form a complete circle, and rather forms a shape not unlike the letter “C.” It is preferred, but not required, that the ribs are spaced one from the next by a distance of between about 0.5 times and about 3 times the thickness of the ribs. Preferably, the ribs are spaced at 1 times the rib thickness. In some embodiments, one or both ends of the protector comprise a termination rib, having a rib width that is greater than the width of the primary ribs. In some embodiments, one or more termination ribs may be interspersed along the length of the protector between the ends to facilitate shortening the length of the protector. In other words, a protector having interspersed termination rings may be shortened at the distal end of the termination ring. Of course, if the termination ring is not needed, any embodiment of a protector can be shortened by cutting the axial spine distally of a semi-circular ring.

One or both ends of the protector may comprise a connector, such as a nut and sleeve combination, to anchor the protector to one or more structures. For example, and not limitation, embodiments of the protector may include a nut and covering sleeve at one end configured to secure the graft to the outflow cannula of a left ventricle assist device. Alternately, the protector may comprise no connectors at either end.

Oftentimes, a vascular graft will need to be removed from a patient, such as when retrieval of a left ventricular device is required. The semi-circular ribs, including primary and termination ribs, if used, are configured to allow removal of the protector from ingrown or surrounding tissue. In some embodiments of the present inventions, the axial spine may comprise an axial channel, preferably disposed about the center of the spine, and configured to guide a scalpel. A scalpel may engage the channel and cut surrounding tissue along the length of the spine. Thereafter, the protector may be removed from around the graft simply by pulling the protector from the ingrown or surrounding tissue. It will now be appreciated that the flexibility of the protector and the semi-circular configuration of the ribs allows the easy removal of the protector. The protector may be fabricated from a wide variety of implantable materials, such as, but not limited to, high density polyethylene (HDPE). It is desired that the material have sufficient flexibility to allow removal of the protector from ingrown or surrounding tissue without damaging the encompassed graft.

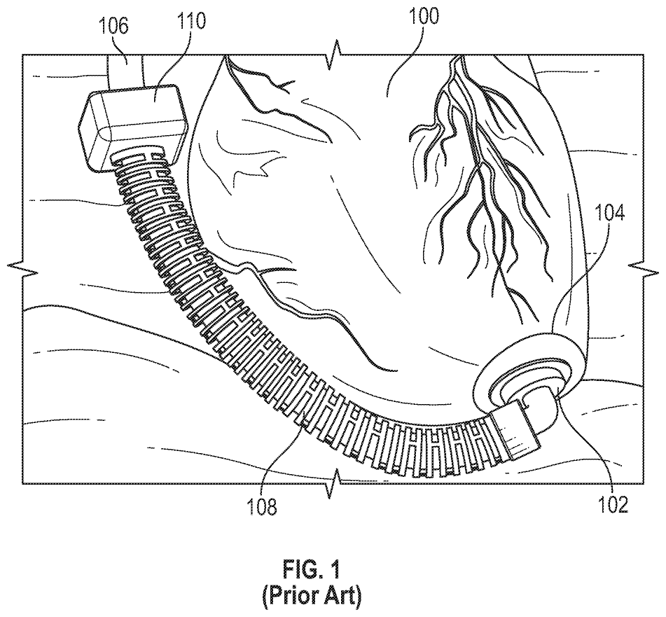

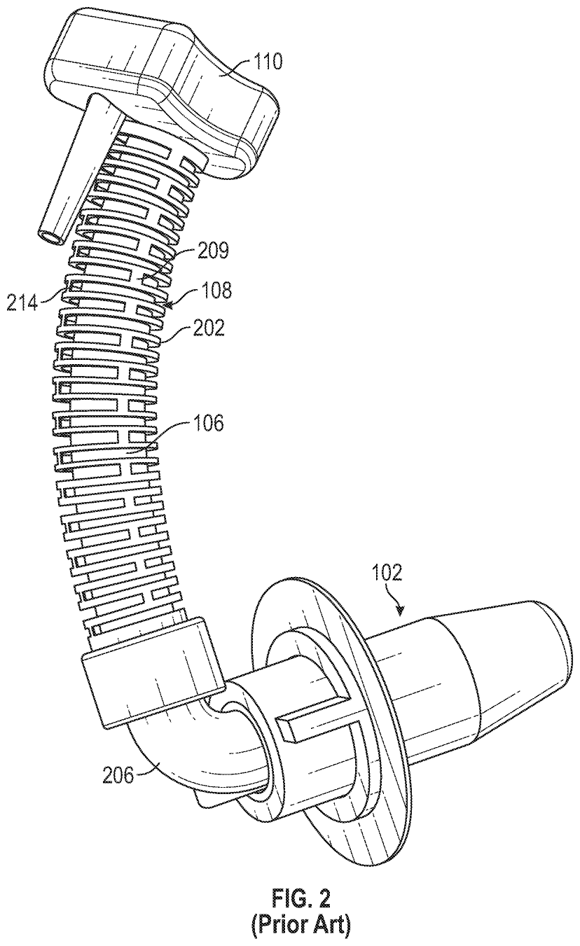

Turning now to the figures, FIGS. 1 and 2 illustrate conventional vascular graft protectors in the context of a left ventricular assist device or LVAD. FIG. 1 illustrates a human heart 100 into which an assist device 102 has been surgically inserted into the left ventricle 104. It will be appreciated that the assist device 102 illustrated is an intraventricular assist device, such as the AVAD™ left ventricular assist device, available from ReliantHeat, Inc. of Houston, Tex. While an intraventricular assist device 102 has been illustrated, the vascular graft protectors disclosed herein are not limited to use with LVADs or cardiac devices generally. It will be understood that a vascular graft 106 connects the intraventricular assist device 102 with the aorta (not shown). A conventional vascular graft protector 108 is illustrated covering, encompassing or substantially surrounding the vascular graft 106 from the intraventricular assist device 102 to a flow probe 110.

As illustrated in FIG. 2, the conventional vascular graft protector 108 comprises interlocked ribs 202. As shown, each rib is interlocked to an adjacent rib by bridges 204. Moreover, the ribs 202 are complete circles, which fully encase, or shroud the vascular graft 106. It will be appreciated that over time, tissue will invade the vascular graft protector 108 structure such that removal of the protector 108 requires extensive surgical manipulation.

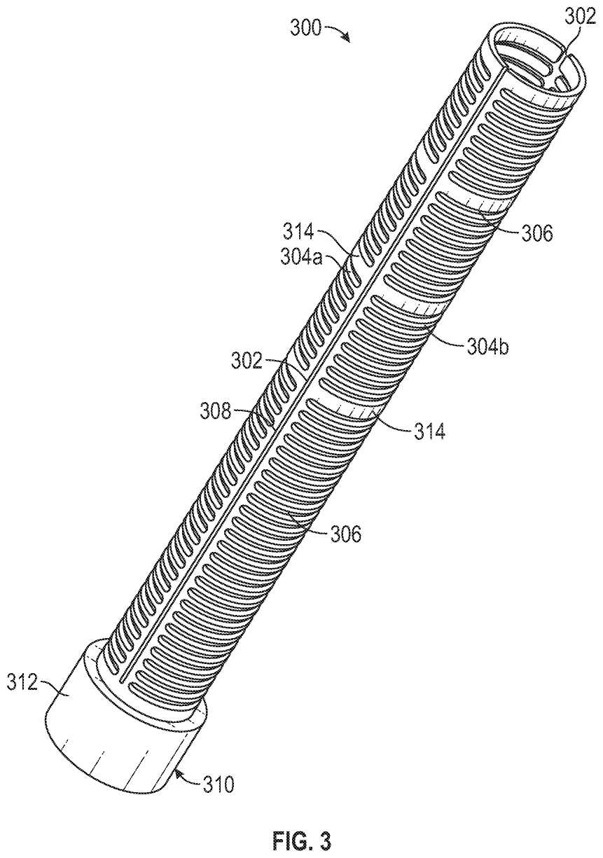

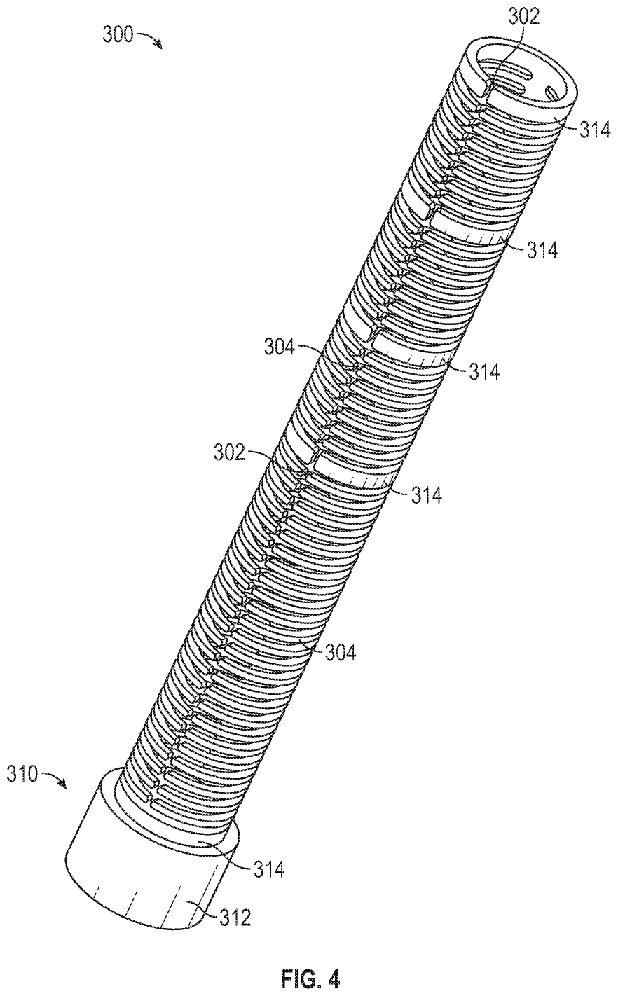

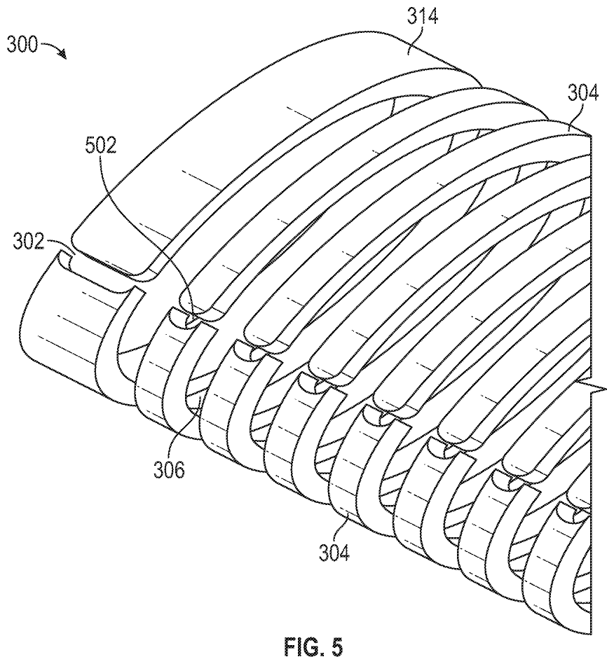

FIGS. 3 and 4 illustrate a vascular graft protector 300 embodying aspects of the present inventions. The vascular graft protector 300 generally comprises a cylindrical body having an axial spine 302 along its axial length. Emanating from the spine 302 may be a plurality of circumferential ribs 302b and 304b. Each rib 304a and 304b form a semi-circular or C-shaped structure (see FIG. 4). In other words the each rib set 304a and 304b do not form a complete circle. As illustrated in FIG. 4, an axial slot separates each rib 304a end from the other rib 304b end.

Each rib set 304 has a rib width, which preferably, but not necessarily, is substantially constant along the length of the protector 300. Each rib set 304 is spaced apart from the adjacent rib set 304 by rib slot 306. As discussed above, it is preferred, but not required, that the rib slots 306 have a width of between about 0.5 times the rib width to about 3 times the rib width. The rib slots 306 increase the flexibility of the protector 300 and allow tissue ingrowth into the protector 300.

Also shown in FIG. 3, is a scalpel guide 308 associated with the axial spine 302. Preferably, the guide 308 is a channel formed in the spine 302, and preferably is centered about the axial spine 302. It will be appreciated that if the protector 300 requires removal, a scalpel can be used to dissect ingrown tissue by running the blade along the guide 308. Once the ingrown tissue along the spine 308 has been dissected, the protector 300 can be removed by pulling the protector 300 free of the surrounding tissue. The semi-circular configuration of the rib sets 304 and the flexibility of the protector 300 allow the protector 300 to be more easily removed than the conventional protectors 108, which have to be cut out of the patient.

Also shown in FIGS. 3 and 4 is connector 310. Connector 310 may comprise a nut (not shown) and a sleeve 312. During the removal of protector 300, the sleeve can be moved away from the nut, the nut undone, and the protector 300 removed from, for example, a device cannula 204 (see FIG. 2). As discussed above, embodiments of the present invention may comprise connectors 310 on each end, on one end, or on neither end.

Other embodiments of the present inventions may comprise one or more termination ribs 314 placed along the length of the protector 300. For example, and not for limitation, each end of the protector 300 may comprise a termination rib 314. Still further, one or more additional termination ribs 314 may be interspersed along the length of protector 300. Termination ribs 314 may be beneficial in connection the protector to structures or components, such as flow probe 110. Also, the overall length of the protector 300 can be adjusted by cutting the axial spine 302 distally of a termination rib 314. It will also be appreciated that the length of the protector 302 can be adjusted by cutting the axial spine 302 distally of a primary rib 304.

Some embodiments of the protector 300 may benefit from a rib interlock system 502. It will be appreciated that one of the purposes of the protector 300 is to prevent or protect the encompassed graft 106 from being crushed or deformed by adjacent organs or body structures. An interlock system 502 allows the ends of a rib set 304a and 304b (or a termination rib set) to structurally interlock and not slide over or past one another. Interlock systems 502 may comprise a convex and concave mating pair, a tongue and groove mating pair, or other mating pair that prevents or minimizes the rib ends from sliding over or past one another.

Other and further embodiments utilizing one or more aspects of the inventions described above can be devised without departing from the spirit of Applicant's invention. Further, the various methods and embodiments of the methods of manufacture and assembly of the system, as well as location specifications, can be included in combination with each other to produce variations of the disclosed methods and embodiments. Discussion of singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to fully protect all such modifications and improvements that come within the scope or range of equivalent of the following claims.

What is claimed is:

1. A vascular graft protector comprising:
a hollow cylindrical body defined by a plurality of sets of semi-circular circumferential primary ribs emanating from an axial spine; and
wherein each primary rib set has first and second ends separated by a circumferential gap.

2. The vascular graft protector of claim 1, further comprising an axial gap separating adjacent primary ribs.

3. The vascular graft protector of claim 2, wherein the axial gap separating adjacent primary rib sets has an axial length of between about 0.5 times and about 3 times a width of the primary rib.

4. The vascular graft protector of claim 2, further comprising a termination rib set at each end of the graft protector wherein each termination rib set has a width greater than a width of a primary rib.

5. The vascular graft protector of claim 1, further comprising a guide channel along the length of the axial spine configured to guide a scalpel.

6. The vascular graft protector of claim 1, further comprising at least one termination rib set located between ends of the graft protector, wherein at least one termination rib set has a width greater than a width of a primary rib.

7. The vascular graft protector of claim 4, further comprising at least one termination rib set located between ends of the graft protector, wherein at least one termination rib set has a width greater than a width of a primary rib.

8. The vascular graft of claim 1, further comprising an interlock system between one or more primary rib sets configured to prevent first and second ends from sliding over or past one another.

9. The vascular graft of claim 1, further comprising an interlock system between each primary rib set configured to prevent each first and second ends from sliding over or past one another.

10. The vascular graft of claim 6, further comprising an interlock system between each primary rib set and termination rib set configured to prevent first and second ends from sliding over or past one another.

11. The vascular graft of claim 7, further comprising an interlock system between each primary rib set and termination rib set configured to prevent first and second ends from sliding over or past one another.

12. A vascular graft protector comprising:
a hollow cylindrical body defined by a plurality of sets of semi-circular circumferential primary ribs emanating from an axial spine;
wherein each primary rib set has first and second ends separated by a circumferential gap;
wherein each primary rib set is separated from an adjacent primary rib set by an axial gap;
one or more termination rib sets located along the length of the protector wherein each termination rib has a width greater than a width of a primary rib;
a guide channel along the length of the axial spine configured to guide a scalpel to dissect ingrown tissue; and
an interlock system associated with one or more ends of primary and termination rib sets and configured to minimize the ends sliding over or past one another.

13. The vascular graft of claim 12 wherein the axial gap is between about 0.5 times and about 3 times a width of the primary rib.

14. A method of removing the vascular graft protector of claim 1 comprising:
dissecting ingrown tissue surrounding the vascular graft protector along the axial spine; and
pulling the vascular graft protector free of the ingrown tissue.

15. A method of removing the vascular graft protector of claim 12 comprising:
dissecting ingrown tissue surrounding the vascular graft protector along the axial spine by sliding a scalpel along the guide channel; and
pulling the vascular graft protector free of the ingrown tissue.
